(19) United States
(12) Patent Application Publication
(10) Pub. No.: US 2011/0269147 A1
(43) Pub. Date: Nov. 3, 2011

(54) Methods, Devices, and Systems for Glycated Hemoglobin Analysis

(75) Inventor: Swetha Chinnayelka, Tarrytown, NY (US)

(73) Assignee: BAYER HEALTHCARE LLC, Tarrytown, NY (US)

(21) Appl. No.: 13/054,543

(22) PCT Filed: Jul. 20, 2009
(86) PCT No.: PCT/US09/51115

§ 371 Date: Apr. 7, 2011

Related U.S. Application Data
(60) Provisional application No. 61/081,799, filed on Jul. 18, 2008.

(51) Int. Cl. G01N 21/75; C12M 1/34

(52) U.S. Cl. 435/7.1; 435/287.2

(57) ABSTRACT

Methods, devices, and systems for measuring the concentration of glycated hemoglobin (HbA1c) in a blood sample are provided. A blood sample may be contacted with a lysing agent, an assay to measure total Hb, and an assay to measure HbA1c, which comprises donor and acceptor dyes attached to a Hb specific antibody and a HbA1c analog. Preferably, the emission spectrum of the donor dye overlaps with the excitation spectrum of the acceptor dye. The concentration of HbA1c in the sample may be correlated with an increase in donor dye and/or a decrease in acceptor dye fluorescence in the presence of HbA1c as compared to the donor and/or the acceptor dye fluorescence in the absence of HbA1c.

### REFERENCE To RELATED APPLICATIONS

This application claims the benefit of U.S. Provisional Application No. 61/081,799 entitled “Methods, Devices, and Systems for Glycated Hemoglobin Analysis” filed Jul. 18, 2008, which is incorporated by reference in its entirety.

### BACKGROUND

Biosensor systems provide an analysis of a biological fluid, such as whole blood, serum, plasma, urine, saliva, interstitial, or intracellular fluid. Typically, biosensor systems have a measurement device that analyzes a sample residing in a sensor strip. The analysis performed by the system determines the presence and/or concentration of one or more analytes in a sample of the biological fluid. In blood samples including hemoglobin (Hb), for example, the presence and/or concentration of total hemoglobin and glycated hemoglobin (HbA1c) may be determined. HbA1c is a reflection of the state of glucose control in diabetic patients, providing insight into the average glucose control over the three months preceding the test. For diabetic individuals, an accurate measurement of HbA1c assists in the determination of the blood glucose level, as adjustments to diet and/or medication are based on these levels.

Conventional methods exist for determining the concentration of HbA1c in a subject. For example, multiple methods based on laminar flow technology are known where reflectance is used to determine the concentration of the total and glycated hemoglobin in a sample. While reflectance may be used, it may lack the sensitivity of fluorescence methods. Furthermore, laminar flow systems may have contamination and other potential problems as the reacted or unreacted portions of the sample must travel over reagents disposed at one or more areas of the laminar flow device. These problems may lead to reductions in accuracy and/or precision for conventional systems. Accuracy may be expressed in terms of bias of the biosensor's analyte reading in comparison to a reference analyte reading, with larger bias values representing less accuracy, while precision may be expressed in terms of the spread or variance among multiple analyte readings in relation to a mean. Bias is the difference between a value determined from the biosensor and the accepted reference value. Thus, simplified methods that provide greater accuracy, precision, and/or sensitivity to HbA1c measurements in clinical and/or at-home settings are needed.

### SUMMARY

The invention provides methods, systems, and kits for measuring the total hemoglobin and HbA1c concentrations in a blood sample, as well as the ratio of HbA1c to total hemoglobin. The HbA1c in the sample may be expressed as a percent of HbA1c/total hemoglobin, for example.

A method for determining the concentration of glycated hemoglobin (HbA1c) in a sample including contacting the sample with a red blood cell lysing mixture; contacting the sample with a total hemoglobin assay; contacting the sample with a HbA1c assay including one of a fluorescent donor dye conjugated to a HbA1c analog and a fluorescent acceptor dye attached to a Hb-specific antibody, and a fluorescent donor dye attached to a Hb-specific antibody and a fluorescent acceptor dye attached to a HbA1c analog, where the emission spectrum of the fluorescent donor dye overlaps with the excitation spectrum of the fluorescent acceptor dye; and correlating the concentration of HbA1c in the sample with a change in the intensity of at least one of donor dye fluorescence and acceptor dye fluorescence.

A hemoglobin ratio determination system including a vial comprising a lysing mixture, a total hemoglobin assay, and a HbA1c assay, the HbA1c assay including a fluorescent donor dye conjugated to a HbA1c analog and a fluorescent acceptor dye conjugated to a Hb-specific antibody, or a fluorescent donor dye conjugated to a Hb-specific antibody and a fluorescent acceptor dye conjugated to a HbA1c analog, and where an emission spectrum of the donor dye overlaps with an excitation spectrum of the fluorescent acceptor dye; and an electronic measurement device including a processor, where the processor determines a ratio of HbA1c to total hemoglobin.

A hemoglobin ratio determination system including a vial comprising a lysing mixture; a sensor strip, where a total hemoglobin assay is included with at least one of the lysing mixture and the sensor strip, where a HbA1c assay is included with at least one of the lysing mixture and the sensor strip, and where the HbA1c assay includes a fluorescent donor dye conjugated to a HbA1c analog and a fluorescent acceptor dye conjugated to an Hb-specific antibody, or a fluorescent donor dye conjugated to a Hb-specific antibody and a fluorescent acceptor dye conjugated to a HbA1c analog, where an emission spectrum of the fluorescent donor dye overlaps with an excitation spectrum of the fluorescent acceptor dye; and an electronic measurement device including a processor, where the processor determines a ratio of HbA1c to total hemoglobin.

A kit for determining the concentration of HbA1c in a sample includes a vial comprising a lysing mixture, a total hemoglobin assay, and a HbA1c assay. The HbA1c assay includes a fluorescent donor dye conjugated to a HbA1c analog and a fluorescent acceptor dye conjugated to an Hb-specific antibody or a fluorescent donor dye conjugated to a Hb-specific antibody and a fluorescent acceptor dye conjugated to a HbA1c analog, where the emission spectrum of the donor dye overlaps with the excitation spectrum of the acceptor dye.

A kit for determining the ratio of HbA1c to total hemoglobin in a sample includes a vial comprising a lysing mixture and a total Hb assay, and a sensor strip comprising a HbA1c assay. The HbA1c assay includes a fluorescent donor dye conjugated to a HbA1c analog and a fluorescent acceptor dye conjugated to an Hb-specific antibody or a fluorescent donor dye conjugated to a Hb-specific antibody and a fluorescent acceptor dye conjugated to a HbA1c analog, where the emission spectrum of the donor dye overlaps with the excitation spectrum of the acceptor dye.

### BRIEF DESCRIPTION OF THE DRAWINGS

The invention can be better understood with reference to the following drawings and description. The components in the figures are not necessarily to scale, emphasis instead being placed upon illustrating the principles of the invention.

FIG. 1 is a schematic of a HbA1c assay.

FIG. 2 is a schematic demonstrating the working principle of the competitive binding of the HbA1c analog and HbA1c to the HbA1c antibody as well as the fluorescence measurements reflecting said competition.

FIG. 3 is a schematic of reflectance and fluorescent measurements of Met-Hb (total Hb) and HbA1c, respectively, from a sensor strip.

FIGS. 4A-4B represent HbA1c analysis kits.

FIG. 5 depicts a schematic representation of a biosensor system that determines an analyte concentration in a sample of a biological fluid.

### DETAILED DESCRIPTION

Improved methods, devices, and systems for analyte analysis in blood samples are described. The methods may be incorporated with devices to provide systems for detecting HbA1c in clinical or home settings. Conventional HbA1c methods, for example, may require a lateral flow of sample through multiple zones on a testing strip or the coating of antibodies on beads, techniques necessitating relatively long analysis times that can introduce errors. Instead, the invention takes advantage of a combination of competitive binding assay chemistry and energy transfer spectroscopic techniques to provide an accurate, simplified, and relatively rapid process for analyzing HbA1c. Surprisingly, a non-enzymatic based competitive antibody/analog binding assay is described that is compatible with energy transfer spectroscopic techniques, such as fluorescence.

FIGS. 1(a) and 1(b) represent assay systems for determining the HbA1c in a blood sample. In FIG. 1(a) blood is placed into a vial containing a blood lysing mixture and a total Hb assay. The lysed sample is then applied to a sensor strip including the HbA1c assay. In FIG. 1(b), the vial also contains the HbA1c assay allowing the sample to react with both assays before being applied to the sensor strip. While not shown in the figures, the sensor strip could include the total Hb assay instead of the HbA1c assay, or the sensor strip could include both assays, where the vial contains the blood lysing mixture, but not either hemoglobin assay.

The blood lysing mixture lyses the red blood cells for the analysis. While the lysing mixture is preferably a solution, the mixture also may be a suspension, a dispersion, a gel, or colloidal. The lysing mixture includes a lysing reagent or reagents compatible with the total Hb and HbA1c assays. Preferable lysing reagents include hemolytic surfactants, such as zwitterionic surfactants. Other useful lysing reagents include cationic, anionic, and neutral species. Examples of cationic species include cetyl trimethyl ammonium bromide, examples of anionic species include sodium dodecylsulfate and sodium deoxycholate, and examples of neutral species include saponin and polyoxyethylene. At present, the zwitterionic surfactant N-hexadecyl-N, N-dimethyl-3-amino-1-propanesulfonate sold as ZWITTERGENT® 3-14 by Roche Applied Science, Roche Diagnostics Corporation, Indianapolis, Ind. is preferred. The amount of lysing reagent for use in the lysing mixture may be selected in response to the volume of sample to be analyzed.

In addition to the lysing reagent or reagents, the lysing mixture may include additional components to dilute the blood sample, buffer the blood sample, stabilize the lysing reagent or reagents, and/or stabilize one or more assays. Non-ionic or ionic surfactants may be used to stabilize the lysing mixture during storage, depending on the nature of the lysing reagent or reagents. Useful non-ionic surfactants include the ethoxylated acetylenic glycols, such as the 400 Series 440, 465 and 485 SURFYNOL® products available from Air Products and Chemicals, Inc., Allentown, Pa. The SURFYNOL surfactants are ethoxylated-2,4,7,9-tetramethyl-5-decyne-4,7-diols having varying ethylene oxide content. For the 400 Series, the ethylene oxide content is varied from 40% to 85% weight/weight (w/w), with the 440, 465 and 485 products having ethylene oxide contents of 40%, 65% and 85% w/w, respectively.

Other useful non-ionic surfactants include block copolymers of ethylene oxide and propylene oxide, such as the PLURONIC® and TETRONIC® lines of surfactants available from BASF Performance Chemicals, Parsippany, N.J. Of these block polymer surfactants, the “L Series” EO-PO-EO type and the “R Series” PO-EG-PO types are preferred. The “L Series” surfactants are polyethylene oxide- polypropylene oxide-polyethylene oxide tri-block copolymers, while the “R Series” surfactants are polypropylene oxide-polyethylene oxide-polypropylene oxide tri-block copolymers.

The amount of nonionic surfactant useful in the lysing mixture can be any amount compatible with the other components of the mixture and the assays. The lysing mixture may include from about 0.001% to about 15% weight of the surfactant per volume of the lysing mixture before addition of the surfactant (w/v). Preferably, the lysing mixture includes from about 0.1% to about 7% w/v of the nonionic surfactant and more preferably from about 0.2% to about 5% w/v of the nonionic surfactant. At present, nonionic surfactant contents from about 0.3% to about 4% w/v are especially preferred in the lysing mixture. Additional information regarding the preparation and content of useful lysing mixtures may be found in U.S. Pat. No. 7,150,995.

The sensor strip may be formed of any transparent material having minimal absorbance in the wavelength region corresponding to the absorbance or transmittance wavelength of the total Hb assay and the excitation and emission wavelengths of the fluorescent donor and acceptor dyes of the HbA1c assay, respectively. The material forming the sensor strip preferably facilitates irradiation of the fluorescent dye or dyes on the strip. Examples of suitable materials from which the sensor strip may be formed include polyethylene terephthalate, polycarbonate, polyvinyl chloride, polyethylene, polyamide, polystyrene, acrylonitrile butadiene styrene, polyester, polyurethane, copolymers of vinyl acetate, vinyl chloride, chloride-silica, paper, nitrocellulose, cellulose acetate, fiber glass, cotton, nylon, silica, agarose, gelatin, fibrous matrices, cross-linked dextran chains, ceramic materials, and combinations thereof.

Once the red blood cells are lysed, total Hb may be determined using any assay capable of determining the total Hb concentration of the sample that is compatible with the HbA1c assay. For example, an assay that converts total hemoglobin to a metal-hemoglobin (Met-Hb) complex, such as described in U.S. Pat. No. 7,150,995, may be used. After lysing, ferricyanide, for example, may be used to form the Met-Hb complex. The intensity of the light responsive to the Met-Hb complex may then be used to determine the total hemoglobin concentration of the sample.

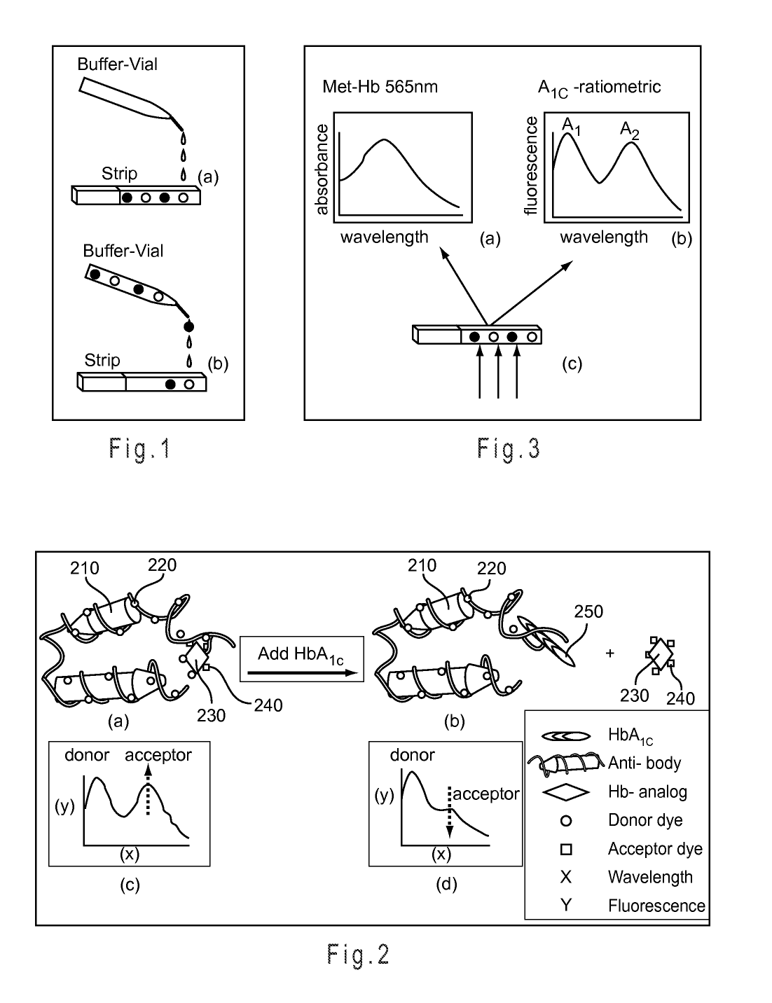

FIGS. 2(a) and 2(b) represent a competitive binding HbA1c assay. FIG. 2(a) represents a HbA1c-specific antibody 210 having an attached first fluorescent dye 220. A HbA1c analog 230 having an attached second fluorescent dye 240 is associated with the antibody 210. Preferably, the fluorescent dyes 220, 240 are attached by conjugation to functional groups available on the antibody 210 and the analog 230, respectively. The fluorescent dyes 220, 240 also may be covalently attached to the antibody 210 and or the analog 230, respectively. While not wishing to be bound by any particular theory, the association between the antibody 210 and the analog 230 is believed attributable to an affinity based mechanism, likely with a reduced affinity due to the modification of the analog.

As represented in FIG. 2(b), in the presence of HbA1c 250, the HbA1c analog 230 dissociates from the antibody 210, thus allowing separation of the first and second fluorescent dyes 220, 240. The HbA1c-specific antibody can be any monoclonal or polyclonal antibody that retains sufficient specificity to HbA1c to provide the desired measurement performance to the analysis.

The HbA1c analog 230 may be any molecule, such as glycated or multiple antigen peptides, which can bind specifically to the HbA1c antibody and has a binding constant lower than that of HbA1c to the antibody. (See for example, Stollner et al., Biosensor Symposium, Tubingen, 2001). Presently preferred glycated peptides for use as HbA1c analogs have an amino acid sequence including some portion of the amino acid sequence of HbA1c. The homology between the HbA1c and the analog may be increased to provide a stronger interaction between the analog and the HbA1c-specific antibody. For example, an analog including the sequence Val-His-Leu-Thr-Pro (VHLTP) may be used. Analogs including other sequences also may be used.

While depicted in the context of HbA1c analysis, the assay may be used for other analytes, such as cholesterol, glutamate, and lactate by switching the antibody to one specific for the analyte of interest and providing an analog that competes with the analyte for the antibody. For cholesterol, an anti-cholesterol antibody may be paired with an analog, such as 22-(N-(7-nitrobenz-2-oxa-1,3-diazol-4-yl)amino)-23,24-bisnor-5-cholen-3β-ol,fluoresterol, NBD-cholesterol, and the like. For glutamate, an anti-glutamate antibody may be paired with an analog, such as glutamate dimethyl ester, alpha-Aminomethylglutarate, and the like. For lactate, an anti-lactate antibody may be paired with an analog, such as benzoylformate and the like. Analogs also may be made by designing appropriate molecular imprinted polymers.

The response of the system may be selected by selecting the dyes attached to the antibody 210 and the analog 230; by altering the labeling ratio of the dyes attached to the antibody 210 versus the analog 230; and/or by selecting the concentration ratio of the antibody 210 to the analog 230. For example, by selecting the specific dyes and/or the labeling ratio of the dyes used with the system, the quantity of light output generated in response to a specific quantity of analyte in the sample may be chosen. Similarly, the sensitivity of the system to a specific analyte may be chosen by selecting the concentration ratio of the antibody 210 to the analog 230. For HbA1c analysis, the system is preferably configured to detect from 0 to 10% HbA1c to total hematocrit (mmol/mmol) in a sample. By preferably configuring the dyes, dye ratio, and antibody/analog ratio, the system can achieve a precision between different assays of ±5%, more preferably ±3%. At present, configurations providing a precision between different assays of ±0.5% are especially preferred.

Fluorescence resonance energy transfer (FRET) can be regarded as the interaction of the transition dipoles of the donor and acceptor dyes. In this phenomenon, when a donor dye is excited at a specific wavelength, the energy is believed to transfer non-radiatively from the donor to an acceptor dye. This transfer occurs when certain criteria are met, including the approximate proximity of the dyes. The distance required between the two dyes for the FRET to occur may depend on multiple factors including the choice of FRET dyes. Preferably, the distance between the donor and acceptor dyes is in the range of about 50 Å to about 100 Å. As the average size of an antibody is about 200 Å and the binding domain size is about 100 Å, a suitable distance of about 100 Å or less may be provided between preferable antibodies and analogues for the formation of FRET capable pairs. Because of the distance requirement, FRET may be substantially reduced or eliminated when the dyes are freely floating in solution.

To measure the dissociation of the analog 230 from the antibody 210, the spectroscopic technique of FRET is preferred, as depicted in FIGS. 2(c) and 2(d). In FIG. 2(c) the complex of the antibody 210 and the analog 230 form strong donor and acceptor peaks due to considerable resonance energy transfer from the donor to acceptor dyes, respectively. Because this energy transfer depends on the distance between the donor and acceptor dyes, when the antibody/analog complex is exposed to HbA1c, the analog 230 is displaced by the HbA1c 250 from the antibody 210 (due to the greater affinity of HbA1c for the antibody). The displacement of the analog 230 from the antibody 210 results in increased distance between the first and second dyes 220, 240. As the dyes separate, resonance energy transfer decreases, thus providing a detectable decrease in the acceptor dye peak relative to the donor dye peak as shown in FIG. 2(d). The donor dye peak also may increase as energy previously adsorbed by the acceptor dye is detected.

While FIG. 2 shows the first fluorescent dye 220 being a donor and the second fluorescent dye 240 as an acceptor, the first dye 220 could be an acceptor and the second dye 240 a donor. Furthermore, although not shown in the figures, one of the dyes could be a quencher, which would result in a reduction of a single emission peak when the analog 230 is associated with the antibody 210. Thus, in addition to donor/acceptor systems where two fluorescence peaks may be observed, in donor/quencher systems one peak is preferably observed. While the terms fluorescent dye or dye are generally used in this application, it is to be understood that in addition to dyes, any species may be used that absorbs and/or emits at desirable wavelengths and is compatible with the assay and sample, including quantum dots and the like. At present, fluorescent dyes are preferred.

A wide variety of fluorescent dyes may be used depending on the desired excitation and/or emission wavelength. In order to avoid the fluorescence from blood and overlap with Met-Hb absorbance, it is desirable that the wavelength of the donor/acceptor dyes be measured in the range of about 450 nm to about 520 nm, or greater than about 590 nm. Examples of suitable dyes include fluorescein isothiocyanate (FITC), ALEXA FLUOR®, and cyan fluorescent protein/yellow fluorescent protein.

Preferred dyes include those sold under the tradename ALEXA FLUOR® by Molecular Probes, Inc. (849 Pitchford Avenue, Eugene, OR 97402-9165 USA). The ALEXA FLUOR dyes are trade secret compositions, with specific examples being Alexa Fluor 488 (AF488) and Alexa Fluor 555 (AF555), for example. Other preferred dyes include the cyanine dyes prepared with succinimidyl ester reactive groups, such as Cy-3, Cy-5, and Cy-5.5. The number immediately after the “Cy” indicates the number of bridge carbons. The number following the decimal point indicates a unique dye structure, which is determined by the substituents on the structure. Cy-3, Cy-5, and Cy-5.5 are available from GE Healthcare, Chalfont St. Giles, UK.

Table I, below, provides examples of suitable donor and corresponding acceptor dyes. The excitation (Exc) and emission (Emm) wavelengths of each dye are provided in nanometers. The excitation wavelength is the wavelength at which the dye absorbs the most light. The emission wavelength is the wavelength at which the dye gives off the most light when excited. The same dye may serve as a donor or acceptor, depending on the choice of the counterpart. For example, TRITC may serve as the acceptor for FITC, but as the donor for Alexa Fluor 647 (AF647). The greater the overlap between the emission wavelength of the donor and the excitation wavelength of the acceptor, the higher the energy transfer efficiency should be from the donor to the acceptor. Preferable donor and acceptor pairs have at least 60% energy transfer efficiency between the dyes, with more preferred pairs transferring at least 80%.

TABLE I

| Donor (Exc/Emm) | Acceptor (Exc/Emm) |
|---|---|
| FITC (488/520) | TRITC (555/575) |
| TRITC (555/575) | AF647 (650/668) |
| TRITC (555/575) | AF660 (665/690) |
| FITC (488/520) | AF568 (578/602) |
| AF568 (578/602) | AF647 (650/668) |
| AF568 (578/602) | AF660 (665/690) |
| AF594 (594/618) | AF660 (665/690) |
| AF594 (594/618) | AF635 (635/648) |
| AF635 (635/648) | AF680 (682/705) |
| AF635 (635/648) | AF647 (647/667) |
| AF647 (647/667) | AF700 (695/720) |
| AF660 (665/690) | AF750 (750/772) |

Table II, below, provides presently favored pairs of donor and acceptor dyes.

TABLE II

| Donor (Exc/Emm) | Acceptor (Exc/Emm) |
|---|---|
| AF594 (594/618) | AF660 (665/690) |
| AF594 (594/618) | AF635 (635/648) |
| AF635 (635/648) | AF647 (647/667) |
| AF647 (647/667) | AF700 (695/720) |
| AF660 (665/690) | AF750 (750/772) |

Unlike FRET processes where both dyes emit, for donor/quencher systems the more efficient the resonance energy transfer between the donor and the quencher, the less light output is measured from the system. Thus, resonance energy transfer may be measured using a fluorescent and a quenching dye as the donor and acceptor molecules, respectively. Any quenching dye may be used that adsorbs light from the donor and is compatible with the analysis. Examples of suitable quenching dyes include dabcyl chromophores and diarylrhodamine derivatives, such as those sold as QSY 7, QSY 9, and QSY 21 by Invitrogen, Carlsbad, Calif. Presently, the diarylrhodamine derivatives are preferred as quenching dyes. When a quenching dye is used, there will not be a substantial second fluorescent peak as a control, unless an additional control and/or reference species is added to the analysis.

FIG. 3(c) depicts a sensor strip irradiated by at least one wavelength of light. The absorbance spectrum reflecting total Hb as Met-Hb is shown on the left in FIG. 3(a), while the dual peak FRET fluorescence spectrum reflecting the HbA1c assay is shown on the right in FIG. 3(b). While the irradiation light is shown passing through the sensor strip in FIG. 3(c), thus having the light source and detector on opposing sides of the strip, the source and detector may be on the same side of the strip or have other arrangements.

From FIG. 3(a), total Hb sample content may be determined by measuring the color intensity/absorbance at about 565 nm or another wavelength compatible with the analysis. Total Hb content also may be determined using transmittance or reflectance at a suitable wavelength. The HbA1c sample content may be determined from ratioing the donor and acceptor peak intensities as shown in FIGS. 2(c) and 2(d). Thus, as HbA1c replaces the analog, the intensity of the donor peak increases as the intensity of the acceptor peak decreases. Larger concentrations of HbA1c in the sample are represented by relatively larger increases in the intensity of the donor peak coupled with relatively larger decreases in the intensity of the acceptor peak. The ratio of the donor and acceptor peaks may provide internal correction for the reactivity of the assay, temperature, and instrument drift, in addition to other factors.

The HbA1c sample content also may be determined from the change in the intensity of the donor or acceptor peak in isolation; however, the benefits provided by using the ratio of the donor and acceptor peaks as an internal indicator of the analysis would be lost. In this regard, one or more species, preferably in the form of one or more fluorescent dyes or quantum dots, may be added to the assay system to provide a comparison for the donor and/or acceptor peaks generated during the analysis.

The HbA1c sample content from FIGS. 2(c) and 2(d) may be compared with the total Met-Hb content from FIG. 3(a) to determine the percent HbA1c in the blood sample. Thus, the HbA1c content of the sample may be expressed as a ratio or percent and may be expressed in terms of mmolar or mg/mL concentrations. Preferably, the analysis can determine HbA1c concentrations in whole blood from about 1% to about 35% (mmolar/mmolar), more preferably from about 3% to about 15% (mmolar/mmolar).

One or more correlation equations relating the peaks measured by the measurement device and the concentrations of Met-Hb and HbA1c in the sample may be obtained by analyzing multiple samples having known analyte concentrations. While multiple calibration techniques may be used, preferably, a three dimensional calibration curve is used, as a different HbA1c curve may be determined for each total Hb concentration. The relationship determined between the known analyte concentrations and their corresponding output signals then may be used to determine experimental sample concentrations from output signals obtained from experimental samples.

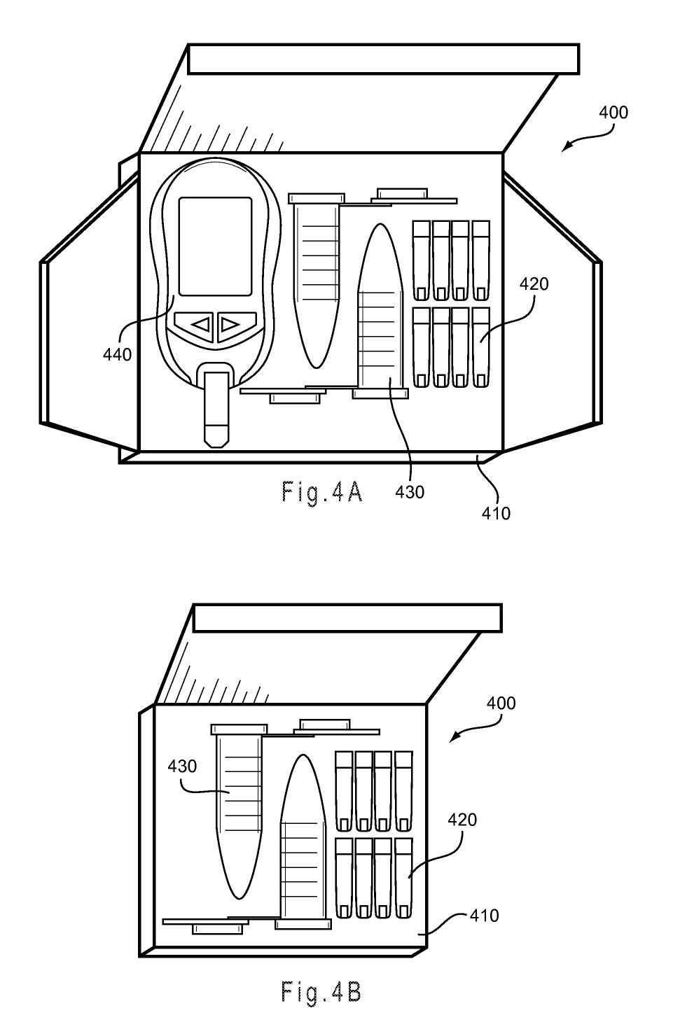

FIGS. 4A and 4B depict cut-away views of HbA1c analysis kits 400. The kit 400 may include an exterior package 410, one or more sensor strips 420, one or more analysis vials 430, and an electronic measurement device 440. The exterior package 410 may have paper and/or plastic components. The exterior package 410 may enclose multiple vials, such as the vials 430, multiple sensor strips, such as the sensor strips 420, the electronic measurement device 440, and one or more supporting structures for the multiple vials, sensor strips, and measurement device usually having paper and/or plastic components, instructions for use, and the like. The supporting structures may be formed from stiff paper, STYROFOAM™, and the like. As an example, the kit 400 of FIG. 4B may be considered as a refill for the kit 400 of FIG. 4A.

The vials may include the red blood cell lysing mixture, the total Hb assay, and the HbA1c assay. Agents omitted from the vials 430, may be included with or without other agents on one or more of the sensor strips 420. The vials may take the form of bottles, ampoules, and the like, which may be formed in part or in whole from plastic, glass, MYLAR®, and the like. The vials may be equipped with fully or partially detachable lids that may initially be part of the vials or may be affixed to the containers by mechanical, adhesive, or other means.

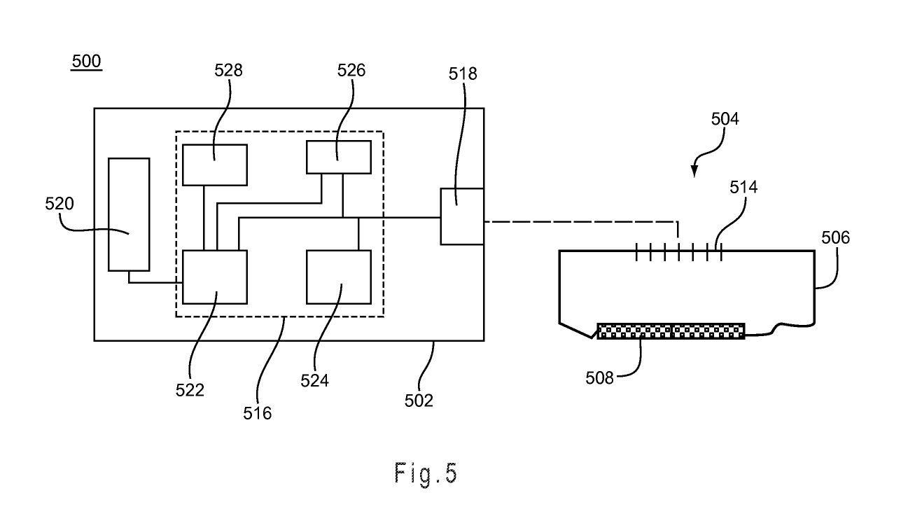

FIG. 5 depicts a schematic representation of a biosensor system 500 that determines an analyte concentration in a sample of a biological fluid. Biosensor system 500 includes a measurement device 502 and a sensor strip 504, which may be implemented in any analytical instrument, including a bench-top device, a portable or hand-held device, or the like. The measurement device 502 and the sensor strip 504 may be adapted to implement an electrochemical sensor system, an optical sensor system, a combination thereof, or the like. The biosensor system 500 may be utilized to determine analyte concentrations, including those of total hemoglobin, HbA1c, lactate, cholesterol, glutamate, and the like. While a particular configuration is shown, the biosensor system 500 may have other configurations, including those with additional components.

The sensor strip 504 has a base 506 that forms a reservoir 508 and a channel 510 with an opening 512. The reservoir 508 and the channel 510 may be covered by a lid with a vent. The reservoir 508 defines a partially-enclosed volume. The reservoir 508 may contain a composition that assists in retaining a liquid sample such as water-swellable polymers or porous polymer matrices. Reagents may be deposited in the reservoir 508 and/or channel 510. The reagents may include one or more enzymes, binders, mediators, antibodies, analogs, and like species. The reagents may include one or more dyes capable of interacting with light. Light includes any suitable electromagnetic radiation from X-ray to infrared. The sensor strip 504 has a sample interface 514 with at least one optical portal or aperture for viewing the sample. The optical portal may be covered by an essentially transparent material. The sample interface may have optical portals on opposite sides of the reservoir 508.

The measurement device 502 includes electrical circuitry 516 connected to a sensor interface 518 and a display 520. The electrical circuitry 516 includes a processor 522 connected to a signal generator 524, an optional temperature sensor 526, and a storage medium 528. The signal generator 524 provides an electrical input signal to the sensor interface 518 in response to the processor 522. The electrical input signal may be used to operate or control the detector and light source in the sensor interface 518. The signal generator 524 also may record an output signal from the sensor interface as a generator-recorder.

The optional temperature sensor 526 determines the temperature of the sample in the reservoir of the sensor strip 504. The temperature of the sample may be measured, calculated from the output signal, or assumed to be the same or similar to a measurement of the ambient temperature or the temperature of a device implementing the biosensor system. The temperature may be measured using a thermister, thermometer, or other temperature sensing device. Other techniques may be used to determine the sample temperature.

The storage medium 528 may be a magnetic, optical, or semiconductor memory, another storage device, or the like. The storage medium 528 may be a fixed memory device, a removable memory device, such as a memory card, remotely accessed, or the like.

The processor 522 implements the analyte analysis and data treatment using computer readable software code and data stored in the storage medium 528. The processor 522 may start the analyte analysis in response to the presence of the sensor strip 504 at the sensor interface 518, the application of a sample to the sensor strip 504, in response to user input, or the like. The processor 522 directs the signal generator 524 to provide the electrical input signal to the sensor interface 518. The processor 522 receives the sample temperature from the temperature sensor 526. The processor 522 receives the output signal from the sensor interface 518. The output signal is generated in response to the reaction of the analyte in the sample. The output signal may be generated using an optical system, an electrochemical system, a combination thereof, or the like. The processor 522 determines total Hb and HbA1c concentrations from the output signals using one or more correlation equation as previously discussed. The results of the analyte analysis may be output to the display 520 and may be stored in the storage medium 528. Communication between the processor 522 and the display 520 may be through wires, wirelessly, and the like.

The correlation equations between analyte concentrations and output signals may be represented graphically, mathematically, a combination thereof, or the like. The correlation equations may be represented by a program number (PNA) table, another look-up table, or the like that is stored in the storage medium 528. Instructions regarding implementation of the analyte analysis may be provided by the computer readable software code stored in the storage medium 528. The code may be object code or any other code describing or controlling the functionality described herein. The data from the analyte analysis may be subjected to one or more data treatments, including the determination of decay rates, K constants, ratios, and the like in the processor 522.

In light-absorption and light-generated optical systems, the sensor interface 508 includes a detector that collects and measures light. The detector receives light from the sensor strip 504 through the optical portal in the sample interface 514. In a light-absorption optical system, the sensor interface 508 also includes a light source, such as a laser, laser diode, a light emitting diode, or the like. The incident beam or beams from the light source may have a wavelength selected for absorption by the reaction product. The sensor interface 508 directs an incident beam from the light source through the optical portal in the sample interface 514. The detector may be positioned at an angle such as 45° to the optical portal to receive the light reflected back from the sample. The detector may be positioned adjacent to an optical portal on the other side of the sample from the light source to receive light transmitted through the sample. The detector may be positioned in another location to receive reflected and/or transmitted light. The detector may include silicone, silicon avalanche, GaAs photodiodes, and like devices capable of converting light into electricity.

The display 520 may be analog or digital. The display 520 may be a LCD, a LED, a OLED, a vacuum fluorescent, or other display adapted to show a numerical reading. Other displays may be used. The display 520 electrically communicates with the processor 522. The display 520 may be separate from the measuring device 502, such as when in wireless communication with the processor 522. Alternatively, the display 520 may be removed from the measuring device 502, such as when the measuring device 502 electrically communicates with a remote computing device, medication dosing pump, and the like.

In use, a liquid sample for analysis is transferred into the reservoir 508 by introducing the liquid to the opening 512. The liquid sample flows through the channel 510, filling the reservoir 508 while expelling the previously contained air. The liquid sample chemically reacts with the reagents deposited in the channel 510 and/or reservoir 508.

The sensor strip 504 is disposed adjacent to the measurement device 502. Adjacent includes positions where the sample interface 514 is in electrical and/or optical communication with the sensor interface 508. Electrical communication includes the transfer of input and/or output signals between contacts in the sensor interface 518 and conductors in the sample interface 514. Optical communication includes the transfer of light between an optical portal in the sample interface 502 and a detector in the sensor interface 508. Optical communication also includes the transfer of light between an optical portal in the sample interface 502 and a light source in the sensor interface 508.

The processor 522 receives the sample temperature from the temperature sensor 526. The processor 522 directs the signal generator 524 to provide an input signal to the sensor interface 518. The sensor interface 518 operates the detector and light source in response to the input signal. The processor 522 receives the output signal generated in response to the total Hb and HbA1c assays as previously discussed. The processor 522 determines the analyte concentration of the sample from the ratio of HbA1c to total Hb, for example. The analyte concentration may be displayed on the display 520 and/or stored for future reference.

While various embodiments of the invention have been described, it will be apparent to those of ordinary skill in the art that other embodiments and implementations are possible within the scope of the invention. Accordingly, the invention is not to be restricted except in light of the attached claims and their equivalents.

1. A method for determining the concentration of glycated hemoglobin (HbA1c) in a sample, comprising:
contacting the sample with a red blood cell lysing mixture;
contacting the sample with a total hemoglobin assay;
contacting the sample with a HbA1c assay including one of
a fluorescent donor dye conjugated to a HbA1c analog and a fluorescent acceptor dye attached to a Hb-specific antibody, and
a fluorescent donor dye attached to a Hb-specific antibody and a fluorescent acceptor dye attached to a HbA1c analog,
where the emission spectrum of the fluorescent donor dye overlaps with the excitation spectrum of the fluorescent acceptor dye; and
correlating the concentration of HbA1c in the sample with a change in the intensity of at least one of donor dye fluorescence and acceptor dye fluorescence.

2. The method of claim 1, where the total hemoglobin assay comprises:
converting hemoglobin to a metal-hemoglobin complex; and
detecting the intensity of light responsive to the metal-hemoglobin complex.

3. The method of claim 1, the fluorescent donor dye having an absorption maximum at an excitation wavelength from about 590 to about 660 nanometers.

4. The method of claim 1, the fluorescent acceptor dye having an absorption maximum at an excitation wavelength from about 630 to about 760 nanometers.

5. The method of claim 1, where the fluorescent donor dye is fluorescein isothiocyanate and the fluorescent acceptor dye is tetramethylrhodamine isothiocyanate.

6. The method of claim 1, where the HbA1c analog is any molecule that binds to the HbA1c-specific antibody with less affinity than HbA1c.

7. The method of claim 1, where the HbA1c analog is selected from the group consisting of glycated peptides, multiple antigen peptides, and combinations thereof.

8. The method of claim 1, where the fluorescence of the fluorescent donor dye and the fluorescence of the fluorescent acceptor dye are detected at a wavelength from about 450 to about 520 nanometers or greater than about 600 nanometers.

9. The method of claim 1, the fluorescent acceptor dye replaced with a quenching dye.

10. The method of claim 1, where the change in the intensity includes an increase in the intensity of the donor dye fluorescence and a decrease in the intensity of the acceptor dye fluorescence.

11. The method of claim 1, where the change in the intensity includes a decrease in the intensity of the donor dye fluorescence and an increase in the intensity of the acceptor dye fluorescence.

12. A hemoglobin ratio determination system, comprising:
a vial comprising a lysing mixture;
a sensor strip,
where a total hemoglobin assay is included with the lysing mixture or the sensor strip,
where a HbA1c assay is included with the lysing mixture or the sensor strip, and where the HbA1c assay includes
a fluorescent donor dye attached to a HbA1c analog and a fluorescent acceptor dye attached to an Hb-specific antibody, or
a fluorescent donor dye attached to a Hb-specific antibody and a fluorescent acceptor dye attached to a HbA1c analog, and where
an emission spectrum of the fluorescent donor dye overlaps with an excitation spectrum of the fluorescent acceptor dye; and
an electronic measurement device including a processor, where the processor is operable to determine a ratio of HbA1c to total hemoglobin.

13. The system of claim 12, where the processor determines a comparison of the decrease in the acceptor dye fluorescence in the presence of HbA1c to the acceptor dye fluorescence in the absence of HbA1c.

14. The system of claim 12, where the processor determines a comparison of the increase in donor dye fluorescence in the presence of HbA1c to the donor dye fluorescence in the absence of HbA1c.

15. The system of claim 13, where the processor determines a correlation of HbA1c in the sample responsive to the comparison and the ratio of HbA1c to total hemoglobin is responsive to the correlation.

16. The system of claim 15, where the processor determines the total hemoglobin in the sample substantially before determining the HbA1c in the sample.

17. The system of claim 12, the fluorescent acceptor dye replaced with a quenching dye.

18. A HbA1c sample concentration determination kit, comprising:
a vial including a lysing mixture, a total Hb assay, and a HbA1c assay, the HbA1c assay including one of
a fluorescent donor dye attached to a HbA1c analog and a fluorescent acceptor dye attached to an Hb-specific antibody, and
a fluorescent donor dye attached to a Hb-specific antibody and a fluorescent acceptor dye attached to a HbA1c analog; and where
an emission spectrum of the donor dye overlaps with an excitation spectrum of the acceptor dye.

19. The kit of claim 18, the fluorescent acceptor dye replaced with a quenching dye.

20. A HbA1c sample concentration determination kit, comprising:
a vial including a lysing mixture and a total Hb assay, and
a sensor strip including a HbA1c assay, the HbA1c assay including one of
a fluorescent donor dye attached to a HbA1c analog and a fluorescent acceptor dye attached to an Hb-specific antibody, and
a fluorescent donor dye attached to a Hb-specific antibody and a fluorescent acceptor dye attached to a HbA1c analog; and where
an emission spectrum of the donor dye overlaps with an excitation spectrum of the acceptor dye.

21. The kit of claim 20, the fluorescent acceptor dye replaced with a quenching dye.
